(19) United States
(12) Patent Application Publication
(10) Pub. No.: US 2022/0084676 A1
(43) Pub. Date: Mar. 17, 2022

(54) SYSTEMS AND METHODS FOR NONINVASIVE DETECTION AND CLASSIFICATION OF CARDIAC FORCING FUNCTIONS MARKERS AND INTERVALS FOR DETECTION OF ABNORMAL HEART CONDITIONS

(71) Applicant: Peter James Neild, Poway, CA (US)

(72) Inventor: Peter James Neild, Poway, CA (US)

(21) Appl. No.: 17/021,734

(22) Filed: Sep. 15, 2020

(51) Int. Cl. G16H 50/20; G16H 10/60; G16H 40/60; A61B 5/00; A61B 5/02; G06N 3/08

(52) U.S. Cl. CPC G16H 50/20; G16H 10/60; G06N 3/08; A61B 5/6803; A61B 5/02; G16H 40/60

(57) ABSTRACT

Heart forcing functions are detected non-invasively using a single sensor mounted vertically at the coronal peak of the upright skull. The sensor mounted with the proper contact pressure on an upright subject can detect the vibratory motion created by the functional movements of the repeating heart-cycle. Acceleration rates during the intervals between the functional moments can be analyzed and with algorithms provide a library of typical heart conditions. The library of marker timing and interval acceleration rates can provide physicians in a clinically relevant manner the origin of recognized heart forcing function conditions, such as valve closings and opening, chamber compressions and relaxations; or allow for the observation of the marker and acceleration intervals for the analysis of unique heart function condition of a particular subject.

### TECHNICAL FIELD

The present disclosure relates to a database engine and techniques for direct entry of disparate, amorphous data and establishment of hierarchical, flat, and custom-defined relationships between these disparate data entries.

### BACKGROUND

Many current methods and systems for detecting problems with a patient's heart are invasive and only collect data on a portion of the heartbeat cycle. Accordingly, it would be advantageous to use noninvasive systems and methods to collect heart information and identify intervals and markers of a heartbeat cycle, and use the collected information to detect abnormal heart conditions.

### SUMMARY OF CERTAIN INVENTIVE ASPECTS

Embodiments of systems and methods are disclosed to detect and identify intervals and markers of a heartbeat cycle based on heart-rate acceleration forces. One innovation includes a system for non-invasively determining information of a condition of the heart. In some embodiments the system can include a first accelerometer adapted to be engaged externally at a position against the top of a patient's head to measure force from acceleration of the patient's skull caused by heartbeat-induced force through the sensitive pathway from heart to coronal peak of the skull, the first accelerometer configured to generate heartbeat force signal data responsive to said acceleration of the patient's upright skull caused by heartbeat-forcing function, the heartbeat force signal data corresponding to one or more heartbeat cycles, and a processing system coupled to the accelerometer. The processing system can be configured to receive the heartbeat force signal data from the accelerometer, determine, based at least in part from the heartbeat force signal data, a plurality of heartbeat cycle markers, heartbeat cycle intervals, and heartbeat force data of each interval, each interval representing a portion of a heartbeat cycle, and determine information relating to a condition of the patient's heart by matching said one or more of the intervals of heartbeat cycle and the heartbeat force data with known signal data of abnormal heart conditions to thereby detect abnormal heart conditions of the patient non-invasively, and generate a display indicative of said abnormal heart conditions of the patient. In some embodiments, the accelerometer communicates the heartbeat force signal data to the processing system via wired or wireless communication means. In some embodiments, the accelerometer includes a storage medium that stores the collected data, and then the data is later communicated to the processing system, either indirectly (e.g., the data is first stored on a computer storage medium and then provided to the processing system) ore directly (e.g., the storage medium of the accelerometer is coupled to the processing system).

Various aspects of such systems can include one or more of the below-described features, or other features described herein. To collect heartbeat force signal data, the accelerometer can be positioned against the top of a patient's head being at the peak and crest of the head of the patient when the head is held in an upright or vertical position. The accelerometer may be positioned against the patient's head with sufficient static pressure to compress the patient's scalp skin and/or hair. The position of the accelerometer is against the patient's head with sufficient pressure that the first accelerometer is considered in contact with the skull. In some embodiments, the accelerometer has a sensitivity to acceleration force of at least 500 mV/G. The processing system is further configured to digitize the received heartbeat force data. The processing system can perform processes to analyze and display information relating to a condition of the patient's heart. For example, the processing system may determine information relating to a condition of the patient's heart includes generating and displaying one or more visualizations on a display that depict one or more of the heartbeat cycle intervals. One for more visualizations may include indications of abnormal conditions of the patient. The processing system may determine the heartbeat cycle intervals based at least in part on determining markers indicting differing rates of change of acceleration trends of heartbeat force data within one heartbeat cycle. Determining information relating to a condition of the patient's heart can include comparing the determined heartbeat force data of at least one heartbeat cycle interval to previously collected heartbeat force data for a corresponding heartbeat cycle interval. The previously collected heartbeat force data may be stored in a database. In some embodiments, the processing system includes a neural network system trained with previously collected heartbeat force data of heartbeat cycle intervals, and the processing system generates an input data set of the heartbeat force data related to the patient and compares the determined heartbeat force data to previously collected heartbeat force data using the neural network system. In various embodiments, the processing system is further configured to identify two, three, four, five, six, seven, eight, nine, ten, eleven, or twelve (or more) heartbeat cycle markers and heartbeat cycle intervals unique to differing heart functions of the heartbeat cycle.

In various embodiments, the processing system system is further configured to analyze said signal data from three or more accelerometers, with one being located at the coronal peak by correlating to signal data from each of said plurality of accelerometers to determine a waveform signal common to the signal data from all of the three or more accelerometers. The processing system can be used to determine intervals of a heartbeat cycle. For example, in some embodiments, the interval of when the heart Atrial Muscle stiffening can be identified by a slow quiescent positive going slope, perhaps with some slight degree of oscillation. In some embodiments, the interval where the heart's atrioventricular (AV) valves closing and Semilunar valve opening coupled with heart muscle contraction can be identified by detecting significant positive going forces which are exerted outwards from the heart to the lungs and body via the aortic arch, in the accelerometer sensitive pathway. In some embodiments, the high positive acceleration changes to that of an extreme negative going recovery, marking the achievement of high blood pressure of the systolic period. In some embodiments, a slow gradual positive rise of acceleration rate can be a basis of continued response and recovery during an interval known as the Mid-Systole Interval. In some embodiments, an interval of quiescent acceleration is exhibited, during which the heart has completed the surge of out-going blood, known as the Late Systole interval. In some embodiments, the atrioventricular valves open and the semilunar valves close coupled with heart muscle relaxation can be identified by detecting an interval when significant positive going forces which are exerted upwards. In some embodiments, the processing system can identify the impact of the AV Valve opening and semilunar valve closing force completion, and a large negative going recovery interval begins, including the structural response of the heart mass not including the damping of contained blood. In some embodiments, an interval of recovery from the large positive and negative acceleration rates can be identified as the early diastole interval, where smaller more damped response oscillations of the heart muscle may be present. In some embodiments, an interval of quiescent slightly negative acceleration can be identified marking the interval called the mid-diastole. In some embodiments, an interval of slightly negative going acceleration can be identified marking the interval called the late-diastole. In some embodiments, a period of slowly positive going acceleration can be identified marking the interval of ventricular filling where blood is enlarging the physical size of the heart with an expanding force. In some embodiments, a period of slowly negative going acceleration can be identified marking the interval where the ventricles have filled to their approximate maximum.

Another innovation includes a system for detecting heart conditions, comprising a processing system coupled to the accelerometer, said processing system configured to receive heartbeat force signal data of a patient, determine, based at least in part from the heartbeat force signal data, a plurality of heartbeat cycle markers, heartbeat cycle intervals, and heartbeat force data of each interval, each interval representing a portion of a heartbeat cycle; and determine information relating to a condition of the patient's heart based at least in part on one or more of the intervals of heartbeat cycle and the heartbeat force data. Such systems can include one or more of the other features described herein.

Another innovation includes a method for non-invasively determining information of a condition of the heart based on heart force information, the method including collecting heartbeat force signal data using an accelerometer adapted to be engaged externally at a position against the top of a patient's head to measure force from acceleration of the patient's skull caused by heartbeat-induced force through the sensitive pathway from heart to coronal peak of the skull, accelerometer configured to generate heartbeat force signal data responsive to said acceleration of the patient's upright skull caused by heartbeat-forcing function, the heartbeat force signal data corresponding to one or more heartbeat cycles, determining, based at least in part from the heartbeat force signal data, a plurality of heartbeat cycle markers, heartbeat cycle intervals, and heartbeat force data of each interval, each interval representing a portion of a heartbeat cycle, and determining information relating to a condition of the patient's heart by matching said one or more of the intervals of heartbeat cycle and the heartbeat force data with known signal data of abnormal heart conditions to thereby detect abnormal heart conditions of the patient non-invasively, and generate a display indicative of said abnormal heart conditions of the patient.

Another innovation includes a method for non-invasively determining information of a condition of the heart based on heart force information, the method comprising receiving heartbeat force signal data, the heartbeat force signal data collected using an accelerometer adapted to be engaged externally at a position against the top of a patient's head to measure force from acceleration of the patient's skull caused by heartbeat-induced force through the sensitive pathway from heart to coronal peak of the skull and to generate heartbeat force signal data responsive to said acceleration of the patient's upright skull caused by heartbeat-forcing function, the heartbeat force signal data corresponding to one or more heartbeat cycles, determining, based at least in part from the heartbeat force signal data, a plurality of heartbeat cycle markers, heartbeat cycle intervals, and heartbeat force data of each interval, each interval representing a portion of a heartbeat cycle, and determining information relating to a condition of the patient's heart based at least in part on one or more of the intervals of heartbeat cycle and the heartbeat force data. The method may further to identify two, three, four, five, six, seven, eight, nine, ten, eleven, or twelve (or more) heartbeat cycle markers and heartbeat cycle intervals unique to differing heart functions of the heartbeat cycle.

Additional embodiments of the disclosure are described below in reference to the appended claims, which may serve as an additional summary of the disclosure.

In various embodiments, systems and/or computer systems are disclosed that comprise a computer readable storage medium having program instructions embodied therewith, and one or more processors configured to execute the program instructions to cause the one or more processors to perform operations comprising one or more aspects of the above- and/or below-described embodiments (including one or more aspects of the appended claims).

In various embodiments, computer-implemented methods are disclosed in which, by one or more processors executing program instructions, one or more aspects of the above-and/or below-described embodiments (including one or more aspects of the appended claims) are implemented and/or performed.

In various embodiments, computer program products comprising a computer readable storage medium are disclosed, wherein the computer readable storage medium has program instructions embodied therewith, the program instructions executable by one or more processors to cause the one or more processors to perform operations comprising one or more aspects of the above- and/or below-described embodiments (including one or more aspects of the appended claims).

### BRIEF DESCRIPTION OF THE DRAWINGS

The disclosed aspects will hereinafter be described in conjunction with the accompanying drawings, which are incorporated in and constitute a part of this specification, and are provided to illustrate and provide a further understanding of example embodiments, and not to limit the disclosed aspects. In the drawings, like designations denote like elements unless otherwise stated or indicated by context of the drawing.

FIG. 1 illustrates certain sensor mounting considerations for data acquisition.

FIG. 2 illustrates an example of a functional block diagram of a sample basic system for the acquisition of data.

FIG. 2a illustrates an example of a functional block diagram relating to data collection using a sensor, a cable, sensor conditioning, signal filtering, analog to digital conversion and communication of the processed signals to another system or to storage.

FIG. 2b illustrates an example of the section of the functional block diagram for input signal conditioning.

FIG. 2c illustrates an example of the section of the functional block diagram for signal filtering of unwanted signal contamination.

FIG. 2d illustrates an example of the section of the functional block diagram for micro-processor controlled digital signal file handling (e.g., communication, processing, storing, etc.).

FIG. 2e illustrates an example of the section of the functional block diagram for data transport and storage, for example, to cloud storage.

FIG. 3 illustrates an example of a top-level view of the application software for the purpose of marker placement and interval determination.

FIG. 4 illustrates an example of an observational analysis function block diagram, showing how determined intervals of a single heartbeat, the single heartbeat from taken from data set of multiple heartbeats.

FIG. 5 illustrates a flow diagram of processing and storing collected heartbeat data.

FIG. 6 illustrates an example of a sample heart-cycle digital data with illustrating placement of markers.

FIG. 7 illustrates an example of positive extreme region of the graphical data, which is the location of the highest acceleration forces.

FIG. 8 illustrates an example of the lowest negative region of the graphical data, which is the location of high recovery regions from the high acceleration forces.

FIG. 9 illustrates middle (low positive and low negative) regions known as the quiescent region of slow or marginal positive and negative acceleration forces to support Claims.

FIG. 10 illustrates an example of Basic Interval Trends without markers or intervals identified to support claims 2 through 16.

FIG. 11 illustrates interval trends without sample data. Each interval is identified with a direction arrow showing likely trend and identification label for alignment with detailed descriptions below and claims.

FIG. 12 illustrates an example of interval “1a—Atrial Muscle Stiffening” with start and end point markers identified and interval trend line shown.

FIG. 13 illustrates an example of interval “1b—AV Close/Semilunar Valve Open” with start and end point markers identified and interval trend line shown.

FIG. 14 illustrates an example of interval “1c—Valve Close/Open Response (Systolic)” with start and end point markers identified and interval trend line shown.

FIG. 15 illustrates an example of interval “1d—Mid Systole” with start and end point markers identified and interval trend line shown.

FIG. 16 illustrates an example of interval “1e—Late Systole” with start and end point markers identified and interval trend line shown.

FIG. 17 illustrates an example of interval “1f—AV Open/Semilunar Valve Close” with start and end point markers identified and interval trend line shown.

FIG. 18 illustrates an example of interval “1g—Valve Open/Close Response (Diastolic)” with start and end point markers identified and interval trend line shown.

FIG. 19 illustrates an example of interval “1f—Early Diastolic” with start and end point markers identified and interval trend line shown.

FIG. 20 illustrates an example of interval “1i—Mid Diastole” with start and end point markers identified and interval trend line shown.

FIG. 21 illustrates an example of interval “1j—Late Diastole” with start and end point markers identified and interval trend line shown.

FIG. 22 illustrates an example of interval “1k—Ventricular filling” with start and end point markers identified and interval trend line shown.

FIG. 23 illustrates an example of interval “1l—Ventricles filled” with start and end point markers identified and interval trend line shown.

FIG. 24 illustrates an example of the likelihood of PVC events by interval location to support possible example of utilization.

FIG. 25 illustrates an example of possible use with response function data with regards to timing and condition recognition. Example used Diastolic Dysfunction.

### DETAILED DESCRIPTION OF CERTAIN INVENTIVE ASPECTS

The detailed description of various exemplary embodiments below, in relation to the drawings, is intended as a description of various aspects of the various exemplary embodiments of the present invention and is not intended to represent the only aspects in which the various exemplary embodiments described herein may be practiced. The detailed description includes specific details for the purpose of providing a thorough understanding of the various exemplary embodiments of the present invention. However, it will be apparent to those skilled in the art that some aspects of the various exemplary embodiments of the present invention may be practiced without these specific details. In some instances, well-known structures and components are shown in block diagram form in order to avoid obscuring various examples of various embodiments.

Although particular aspects various exemplary embodiments are described herein, numerous variations, combinations and permutations of these aspects fall within the scope of the disclosure. Although some benefits and advantages of certain aspects are mentioned, the scope of the disclosure is not intended to be limited to particular benefits, uses or objectives.

Some systems, for example, as described in U.S. Pat. Nos. 10,076,274 and 8,905,932, depict a methods and systems that are focused on the response to vibratory motion of the human brain, without consideration or analysis of the forcing functions that create the studied responses, other than timing considerations. Such systems are designed to focus on the response signals of the vibratory response functions of particular neurological and brain physiological medical conditions and includes emphasis to eliminate the contributions of other signal sources, respiration and body/head movement, that are considered distractions.

While such systems acknowledge that many of the inter-cranial response functions are stimulated by blood flow into the brain there is no acknowledgement of the particular forcing functions created by heart function acceleration forces. Such systems do not address the force portion of the natural transfer function, nor is there any consideration for obtaining that information. Instead, the system simply ignores the input portion of the basic transfer function equation, which is one of the focuses of this disclosure.

Using the systems and methods described herein, heart forcing functions can be detected non-invasively using a single sensor mounted vertically at the coronal peak of the upright skull. The sensor can be, for example, an accelerometer. In some systems, more than one sensor is used, each of the sensors being mounted vertically at the coronal peak of the upright skull. The one or more sensors can be the same, or have difference sensitivities/sensing characteristics.

A sensor mounted with the proper contact pressure on an upright subject can detect the vibratory motion created by the functional movements of the repeating heart-cycle. Acceleration rates during the intervals between the functional moments can be analyzed and with algorithms provide a library of typical heart conditions. The library of marker timing and interval acceleration rates can provide physicians in a clinically relevant manner the origin of recognized heart forcing function conditions, such as valve closings and opening, chamber compressions and relaxations; or allow for the observation of the marker and acceleration intervals for the analysis of unique heart function condition of a particular subject.

In one example, a system for non-invasively determining information of a condition of the heart can include a single accelerometer adapted to be engaged externally at a position against the top of a patient's head to measure force from acceleration of the patient's skull caused by heartbeat-induced force through the sensitive pathway from heart to coronal peak of the skull. In other examples, the system can include two or more accelerometers positioned near each other to each measure force from acceleration of the patient's skull caused by heartbeat-induced force through the sensitive pathway from heart to coronal peak of the skull. In some instances, the two or more accelerometers provide for redundancy of data. For example, the two or more accelerometers may have the same sensitivity to detecting acceleration or force. In other examples the two or more accelerometers may have different sensitivity for detecting acceleration or force. The accelerometer is configured to generate heartbeat force signal data responsive to said acceleration of the patient's upright skull caused by heartbeat-forcing function, the heartbeat force signal data corresponding to one or more heartbeat cycles. The system can include a processing system coupled to the accelerometer, said processing system configured to receive the heartbeat force signal data from the accelerometer, and determine, based at least in part from the heartbeat force signal data, a plurality of heartbeat cycle markers, heartbeat cycle intervals, and heartbeat force data of each interval, each interval representing a portion of a heartbeat cycle. The processing system can also determine information relating to a condition of the patient's heart based at least in part on one or more of the intervals of heartbeat cycle and the heartbeat force data. Such embodiments and other embodiments are further described below. Additional information is provided in Appendices A, B, and C of this disclosure.

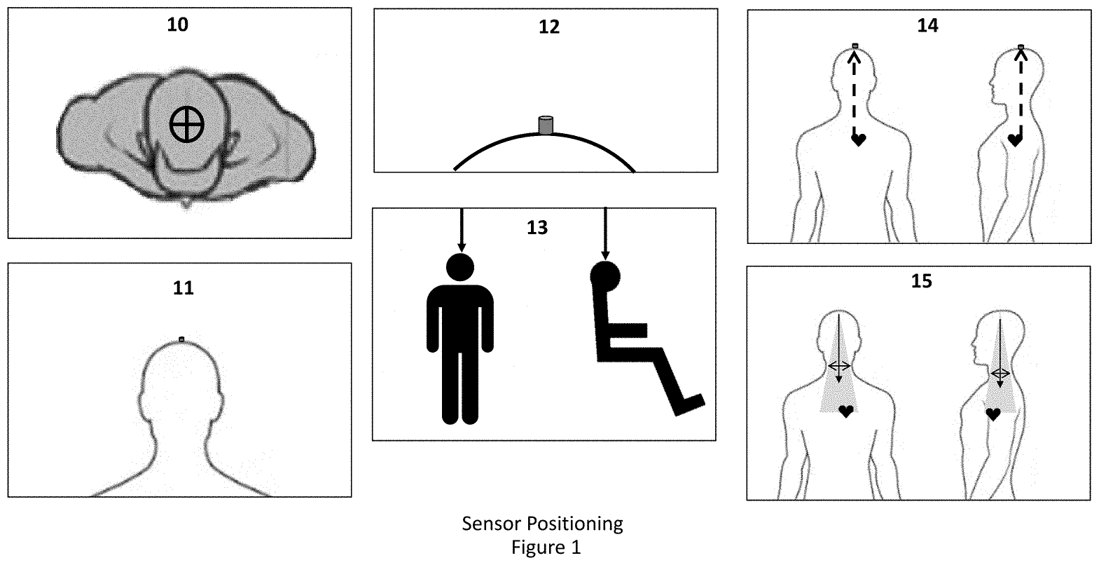

FIG. 1 shows the critical orientation of the accelerometer sensor location [10]. The sensor positioning and mounting is the critical aspect of this method's implementation. The Coronal sensor must be mounted absolutely at the top of the subject's head [11] on the peak of the crest [12] with the subject sitting or standing absolutely upright [13], such that it can detect the vertical acceleration forces created by the heart's activity [14]. It must be understood by the operator and the subject that body positioning and stillness is a critical aspect. Sensors properly mounted to the subject's skull without this absolute vertical (normal earth G force orientation) will have much larger response signals from other sources present in the data. Whereas absolute vertical will have the emphasis placed on the target heart force for this method to be effective [14]. The sensor must have a factor of cross axis rejection [15] at or nearing 10° off axis such that most cross axis acceleration forces from other sources are rejected by the sensor itself. Additionally, the sensor must be oriented such that the center-line sensitivity axis is pointed directly down the center of the subject's neck aimed at the aortic arch [14], which is a relatively large mass exhibiting the positive and negative acceleration forces propagated from the heart function. Finally, the sensor must have a sensitivity of at least 500 mV/G where the mounting position will provide the nominal earth surface 1G of static attraction, as the “zero” point for the desired positive and negative vertical acceleration forces. It is imperative for this method to be effective that the three critical factors be adhered to as they are paramount to the method's novelty. Sensor Position, Sensor Orientation, and Sensor sensitivity axis must be maintained for the success of the method.

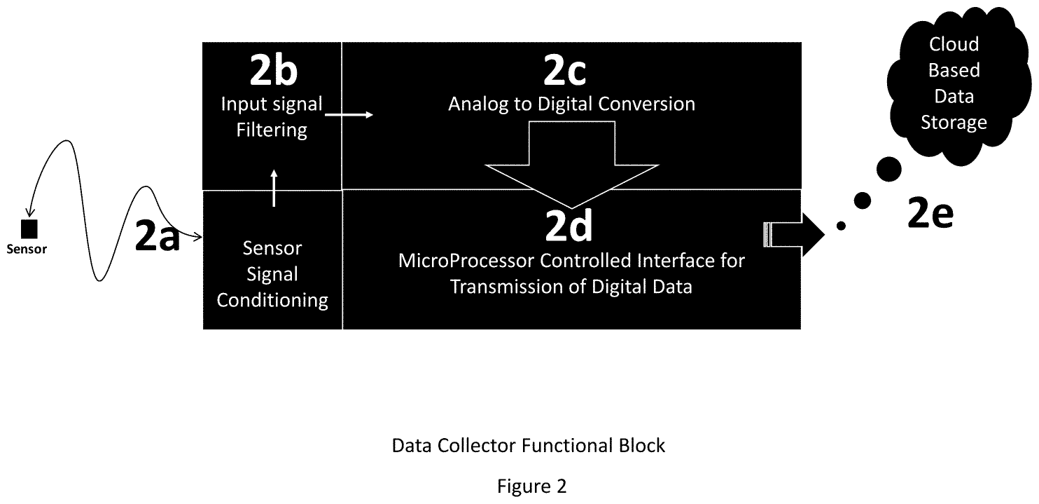

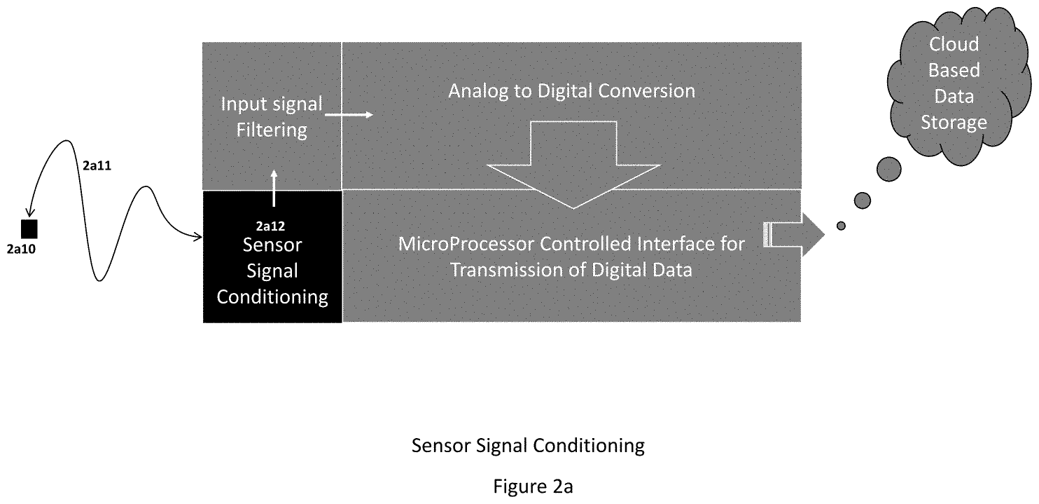

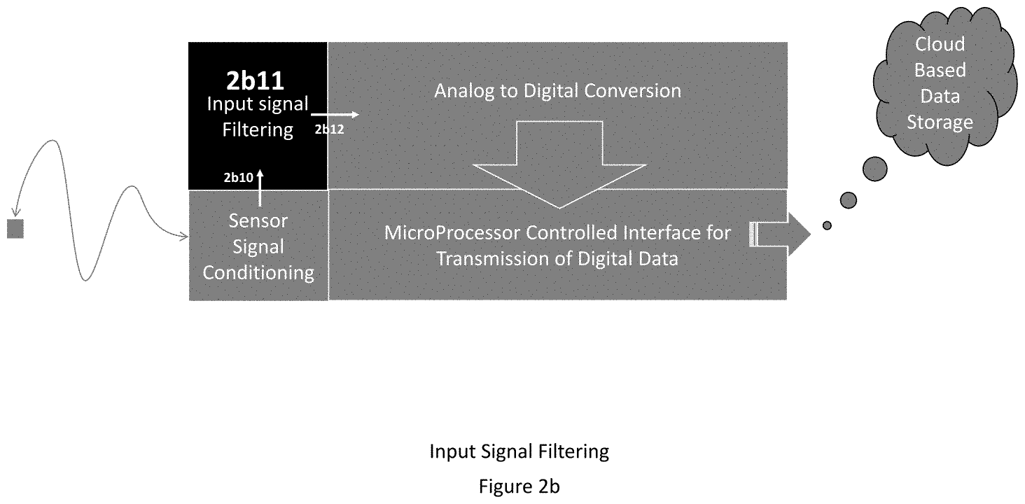

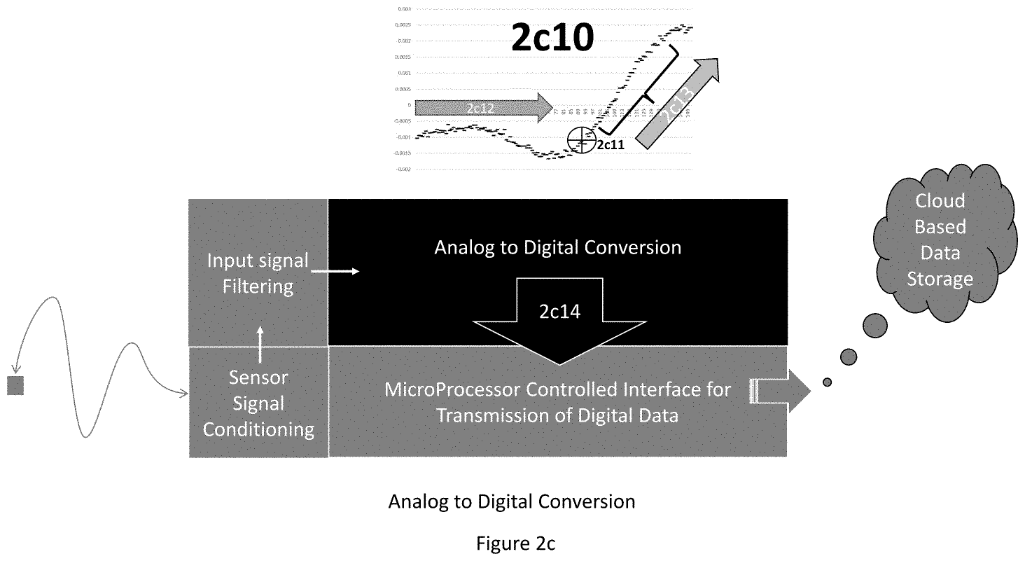

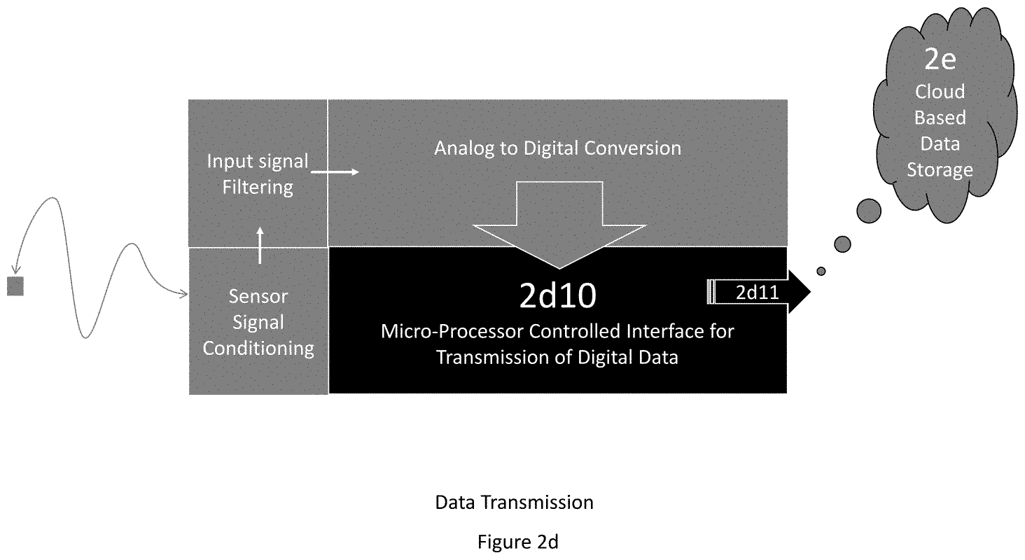

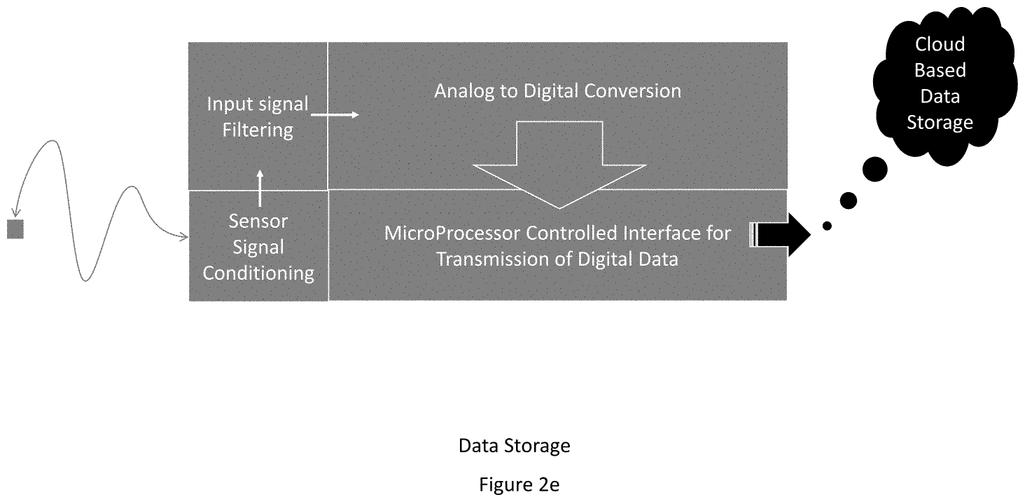

FIG. 2 shows a basic functional block diagram of the method's data collection system. While again the system as configured has other applications, there are particular aspects of this method that are unique. FIGS. 2a through 2e place emphasis on each stage for the descriptions of the critical aspects for this method.

FIG. 2a shows the sensor itself [2a10], the connection to the system [2a11] and the very first electronic section of the data collection system[2a12]; the Sensor Signal Conditioning. This section of the system must preserve and place emphasis on the near Direct Current (DC) component of the received signal's characteristics. Again, from paragraph 0044 the sensor's DC component represents the static 1G pull of earth's gravity upon the subject as neutral. Positive and Negative acceleration forces are those created by the heart's functional forces, and they must be received without distortion, or attenuation.

FIG. 2b shows the analog transfer [2b10] of the properly conditioned data from the sensor and its critical location and position such that all low frequency and gradual changes of acceleration forces are preserved. Analog signal integrity is required. Similarly, the second stage of the system [2b11], the Input signal Filtering used for this method must also preserve these signals. However, higher frequency signals must be filtered out, without distorting the low frequency and near DC signals that are the heart function acceleration forces, en-route to the next stage [2b12] analog signal integrity.

FIG. 2c highlights the Analog to Digital conversion process, where the acceleration signal is replaced by a digital representation (150 sample points shown in the inset [2c10]) of discrete levels of acceleration for individual points in time. The interval of time must allow for the discrete identification of specific points in time where markers will be placed. The placed markers will then define the heart function intervals. The data shown in the inset [2c10] is the sampled data of where the AV Valve Close/Semilunar Valve Open starting marker will be placed [2c11], which shows the end of the Atrial Muscle Stiffening interval [2c12] and the beginning of the Systolic rapid acceleration [2c13] created by the valves open/close transition and blood flow into the aortic arch. It can easily be seen that the highly accurate Analog to Digital conversion allows for the required precision with the individual sample points represented as very small intervals of time ready for transport [2c14] to the next section as a digital description of the data.

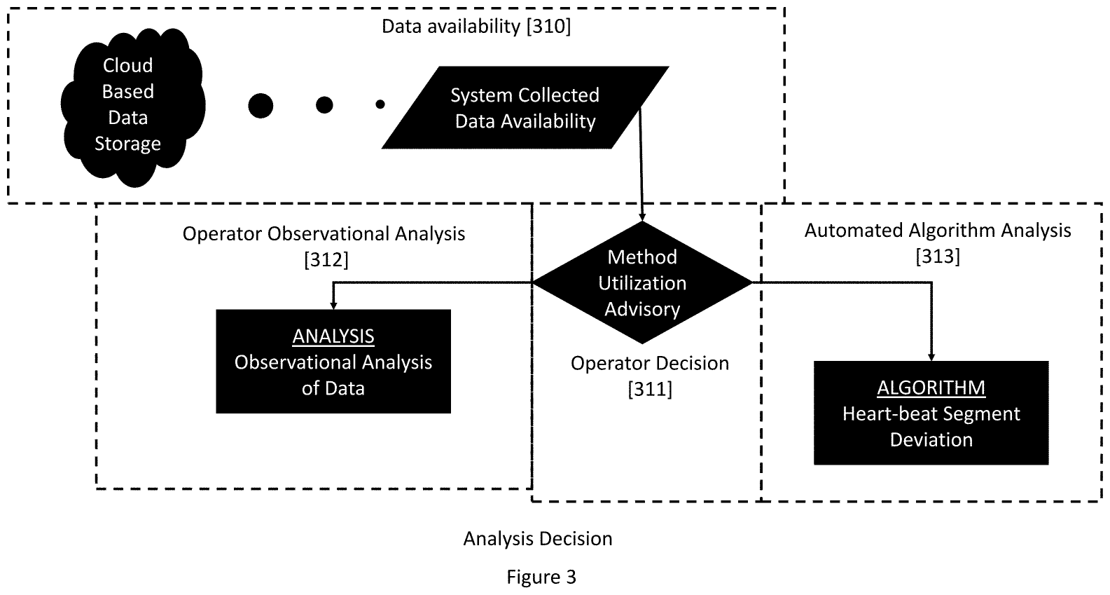

FIG. 2d presents the micro-processor-controlled interface [2d10] where data from the Analog to Digital Converter is partitioned into data sets that represent the data collected during the measurement session. Proper file identification is provided and the data is organized for transmission [2d either directly to the cloud-based Data Storage system [2e], for later retrieval, or to visual displays of the data collection attendant software application. Paragraphs 90 through 105 provide more information about this particular section of the block diagram, along with FIGS. 3, 4 and 5 with their operational descriptions 0057 through 0065.

FIG. 3 is a basic functional block diagram showing that the collected data residing on a Cloud-based Data Storage organized by file name structure is available [310], which in turn can take one or both of two paths [312] or [313]. The normal and automatic utilization will be to an Algorithm based software application [313] that automatically selects individual heart-cycle data sets and places in them the markers for the different heart forcing functions, dividing the cycle into those different intervals for analysis and diagnosis probabilities. The operator also has the choice [311] of performing observational analysis and comparison [312] to either the raw data collected or in comparison with the algorithm results from current or historic data [313].

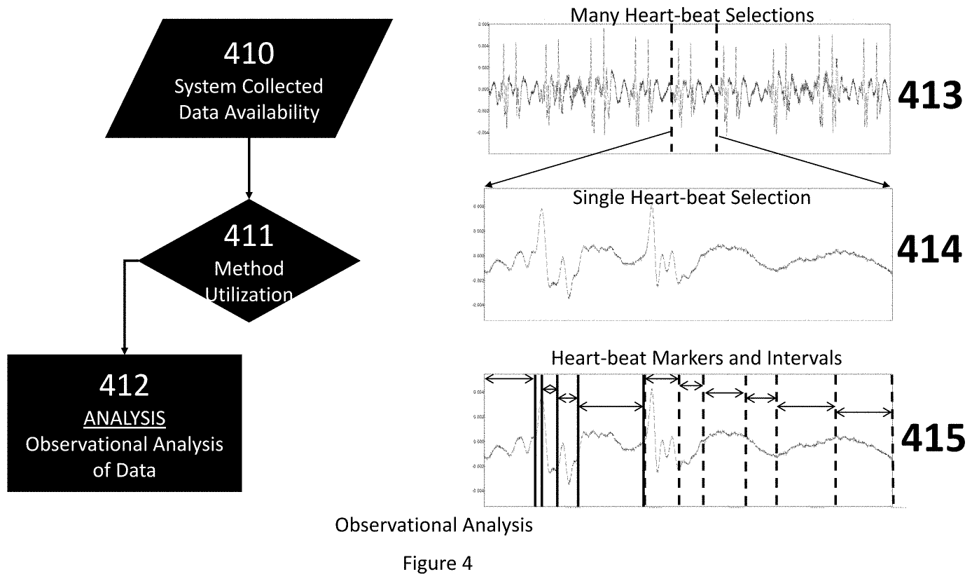

FIG. 4 shows the work flow of one of the two choices [411], given that data exists [410], that of Observational Analysis [412], where an operator may view the entire set of collected data, consisting of many contiguous heart-cycles [413]. The software application provides observational enhancing tools and the ability to scale the data [414] for observation or the manual placement of markers [415] creating intervals within a single heart-cycle [414]. Additionally, the operator may choose to review algorithm created markers and intervals and compare them to those manually created and save the resultant data for future use.

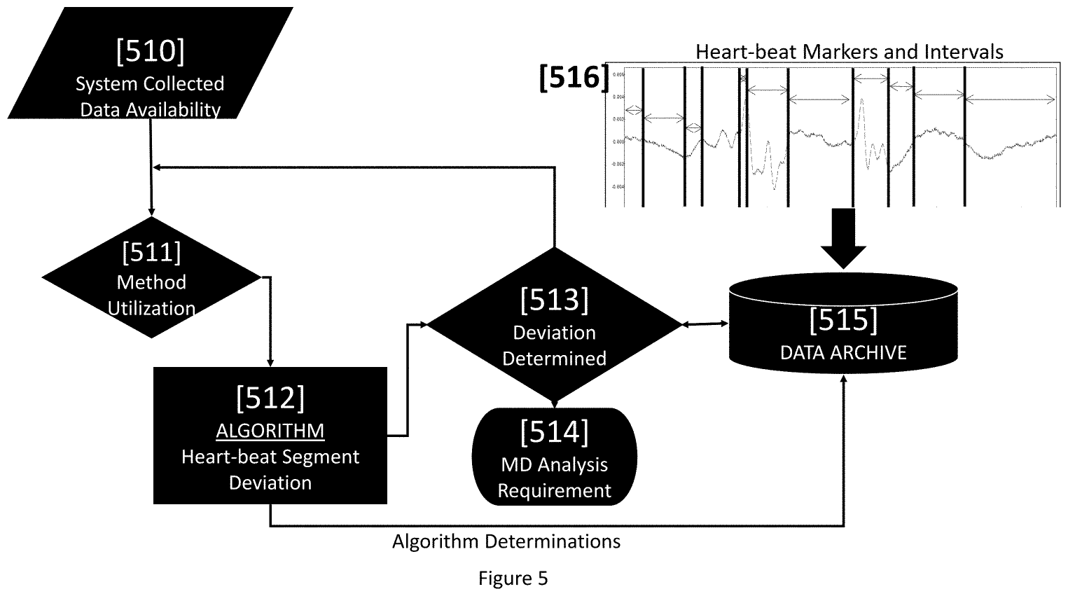

FIG. 5 provides a view of the second of the two choices that actually takes place automatically upon the receipt of a new data file [510] or at the choice of the operator [511]. The algorithm portion [512] of the application performs comparisons [513] to existing files with markers and intervals already established [514] and provides information on deviations to those files. If the deviations are medically significant a notification is provided [515] to the operator stating that a comparison to a previously recorded condition has been recognized, along with statistical parameters of the deviation [515] comparison such that the operator can elect the proper action to be taken. If the measured data is not significantly deviated from normal marker and interval timing and shapes, then the statistical values of those parameters are adjusted and made available to the operator for observation [516] should they choose to do so.

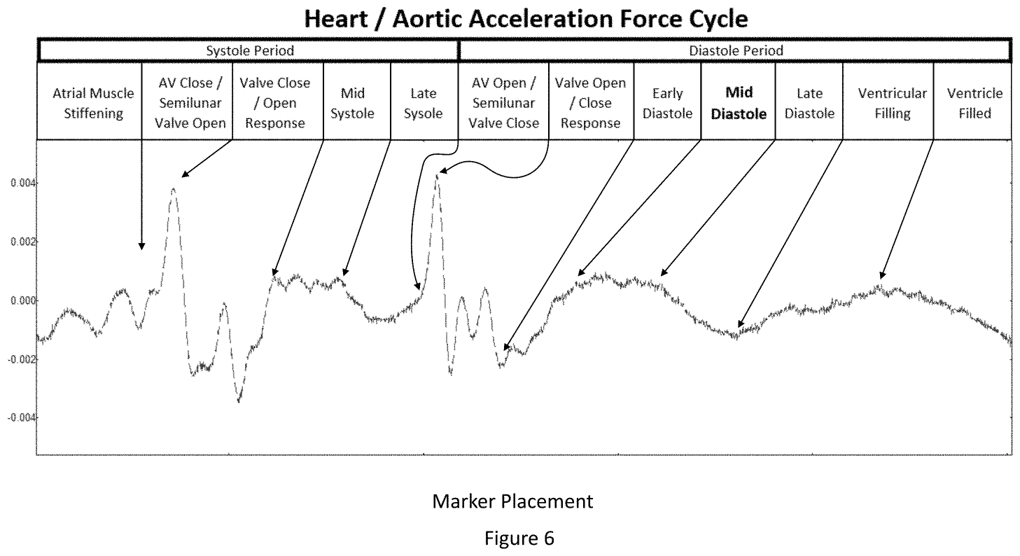

FIG. 6 illustrates the knowledge to date with approximate markers and intervals within one human heart-rate cycle shown. Markers will be placed in the approximate area of each arrow; each at a point where the acceleration trend by definition changes. The intervals between two selected markers become the named intervals. The normal “average” data base is under development with the system, along with deviation statistics. It is fully expected that more heart-function forces will be identified as the system is utilized, and as the statistical counts increase additional markers and intervals will be added to the standard.

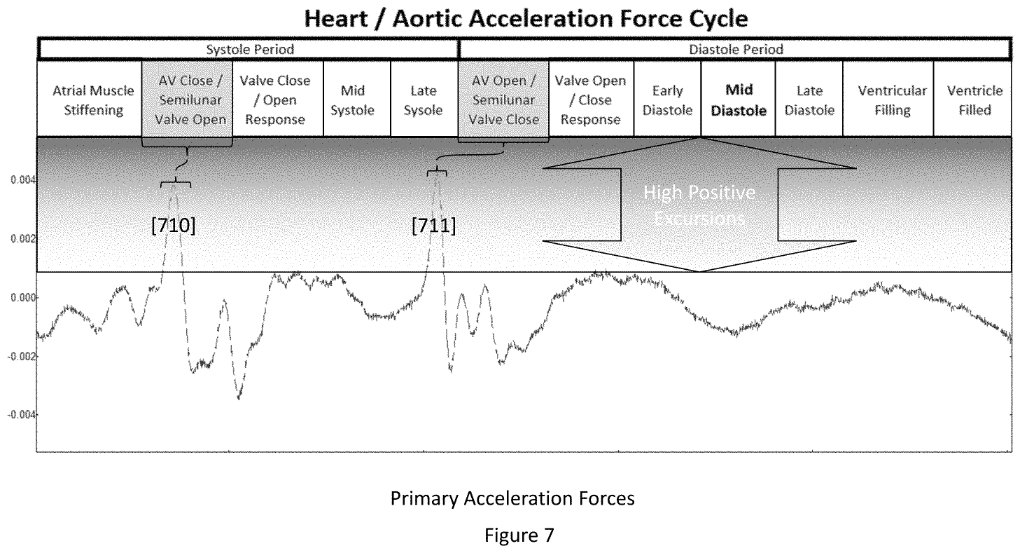

FIG. 7 provides the plotted region for signal analysis that matches that provided by stethoscope utilization. The relatively high positive excursions provide indications of Systole [710 ] and Diastole [711] strength of force and timing relationships within the cycle, and are easily related to that of the stethoscope predominant sounds. Irregular shapes to these two dominant positive forcing functions in shape or timing or additional large positive forces in this highlighted region provide indications of deviations for examination.

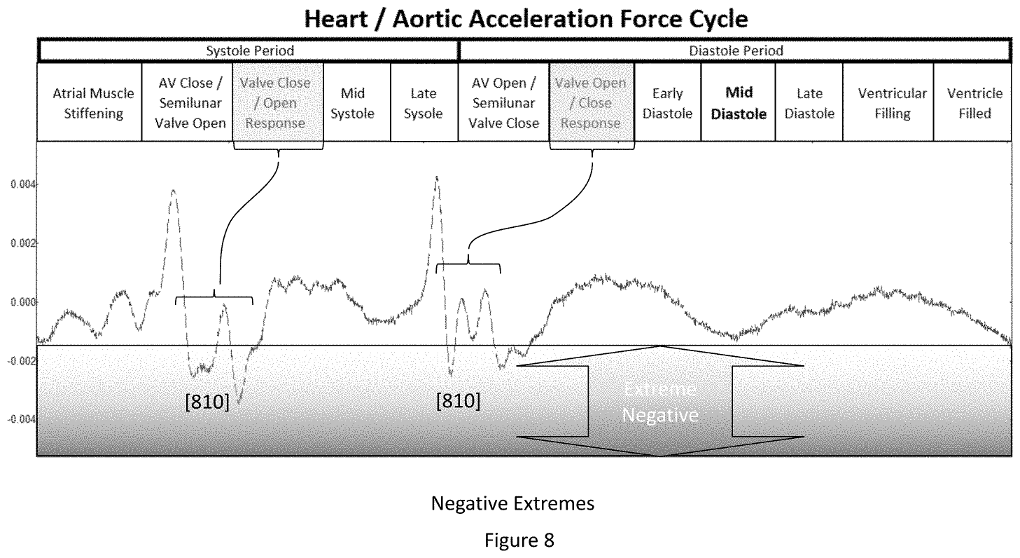

Similarly, in FIG. 8 the extreme negative going forces [810] and [811] that are associated with the passing of the primary positive going forcing functions are also examined for marker and interval placement and potential deviations from normal. This is especially true of strong negative deviations that are not associated with positive going forces.

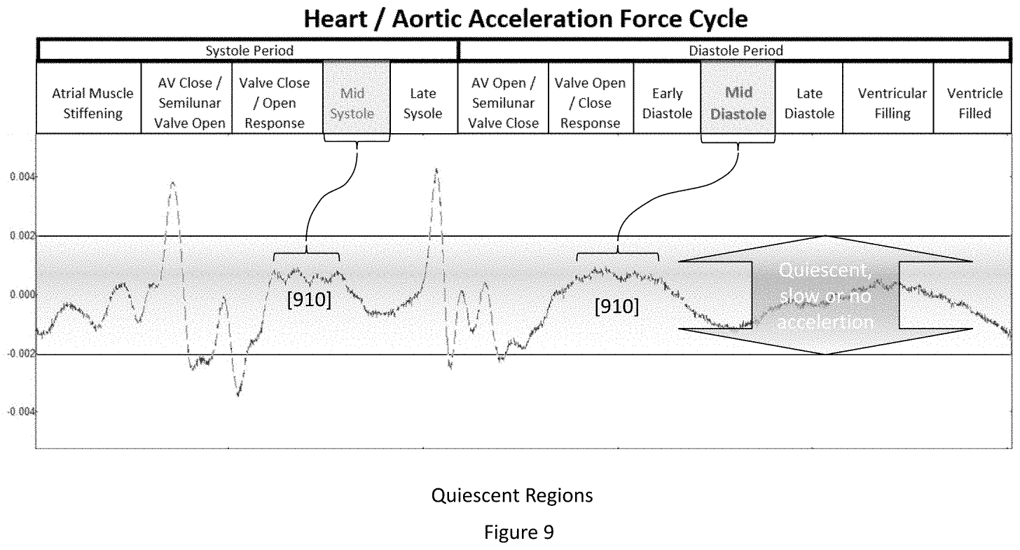

In FIG. 9, the quiescent slow rates of positive and negative going acceleration rates making up the bulk of the heart-cycle intervals are examined. It is important to note the near flatness or lack of acceleration forces during the Mid-Systole [910] and Mid-Diastole [911] intervals. These particular intervals may or may not be present, and are most likely not present in high heart-rate measurement sessions.

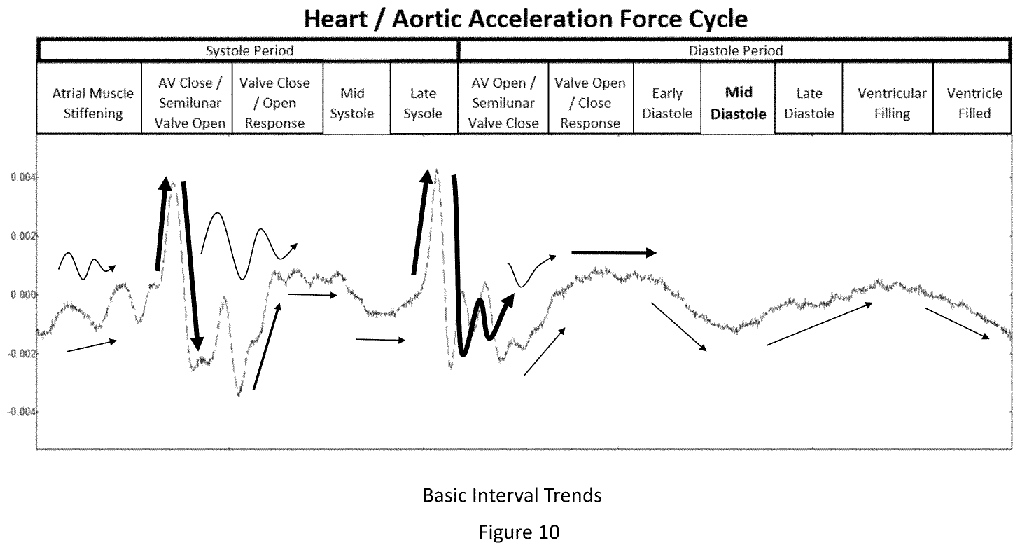

FIG. 10 shows the collection of markers and intervals present in the three regions (positive/negative and quiescent) that currently provide 12 marker locations and derived intervals that far surpasses the 5 markers available via the second passive system, that of ECG or EKG representations of the P, Q, R, S and T.

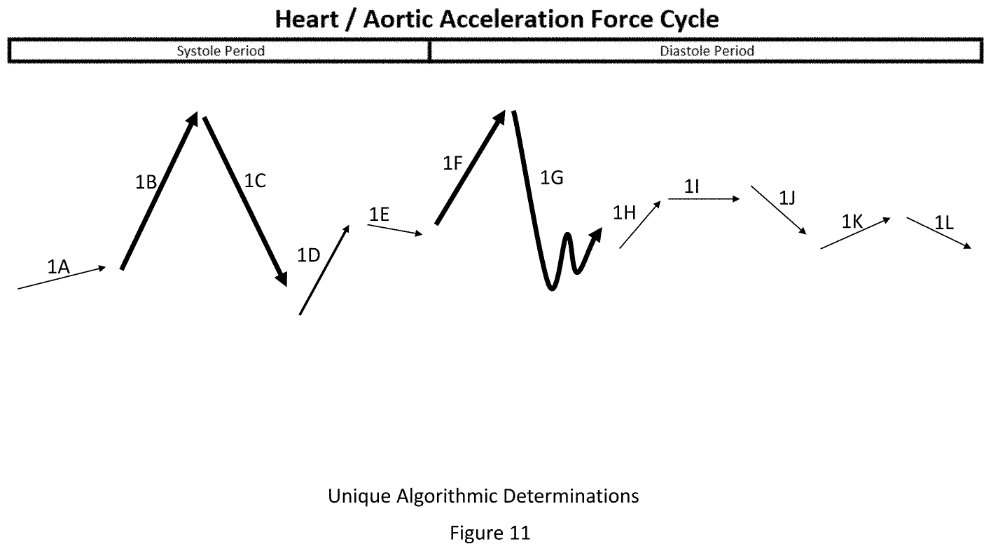

FIG. 11 Illustrates approximate examples of the basic “trends” of the 12 intervals currently identified with specific heart forcing functions. As stated earlier more markers and intervals will be added over time as the system is utilized adding to the statistical counts and deviations. In FIG. 10 the often difficult to ignore response oscillations are shown in their most likely intervals above the actual data trace, whereas the actual force trends are shown below the actual data trace. Well trained operators or algorithms with sufficient statistical deviation populations will “see” past the distractions of oscillatory response signal impact upon the force.

There are two areas of claims made possible by this method. The first set (numbered 1A though 1L) have to do with the placement of markers and intervals, and the second (2) where existing systems focused on intercranial acceleration responses would be enhanced with the implementation of the heart functional forces characterizations.

The heart is a muscular organ consisting of four chambers, functionally separated from each other with valve tissue. The heart is located typically just behind the centerline of the breastbone and slightly to the left of center, where the aortic arch is located above and more central for the distribution of the blood forced out by the heart muscle contractions. The four chambers are known as, the right atrium, the right ventricle, the left atrium and the left ventricle. The left ventricle is considered the strongest chamber which pumps oxygen rich blood to the body. The acceleration force created by the heart contraction creates dynamic blood pressure, which completes the cycle of blood flow. It is also important to note that the right ventricle pumps blood to the lungs where it is loaded with the oxygen for transport through the body by the aforementioned left atrium and ventricle.

The method of detection and classification has identified the following intervals, each having general trends of acceleration that are unique and easily differentiated from each other. The instantaneous transition from each interval to the next create the existence of markers, such that the timing of the intervals can be compared and analyzed for the detection of heart abnormality or malfunction. The intervals and markers are shown in FIG. 11; and described here as the basis for the claims, each interval (A through L) is shown in FIG. 11.

The claims are organized in accordance with prior medical devices that fall short of the improved resolution provided by this method. The first step in the organization is that provided by the usage of stethoscopes which provided the first method of heart-cycle analysis into two parts; the systolic interval and the diastolic interval. It is important to note that the method of detection used by the invention of the stethoscope was that of acoustic analysis where the two acoustic “signals” were produced by the two large primary acceleration forces created by the heart shown in FIG. 7. The second step in the organization of this method's claims comes from the P, Q, R, S, T sequence provided by the invention of the electrocardiograph. The P, Q, R, S, T are five points in time of electronic signals provided to the heart by the nervous system controlling the sequence of intervals identified by this method, within the two provided by the stethoscope. Finally, it is to be understood that future analysis and algorithm development using this method will in all likelihood further refine the markers and intervals listed here with additional markers and intervals that will be considered sub-interval markers and intervals to those listed here alpha-numerically:

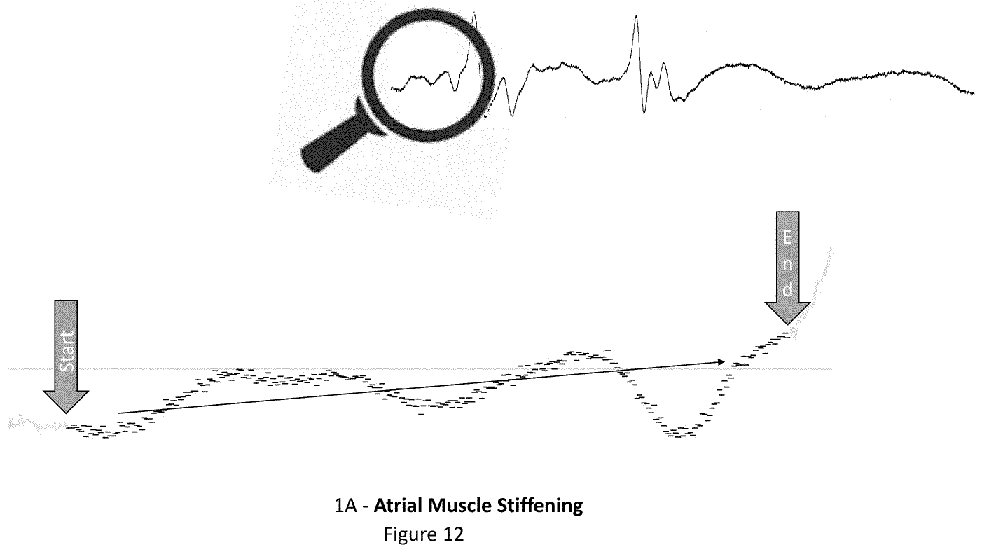

1A—Atrial Muscle Stiffening (FIG. 12)—The interval of Atrial Muscle stiffening follows after the interval where the Ventricles are Filled. The acceleration trend is slightly positive-going within the slight negative region where acceleration is considered near quiescent shown in FIG. 9. The slow positive rise of this interval will terminate with the first (Systolic) large positive acceleration force created by contraction of the ventricles. It is not uncommon for the acceleration trend of this interval to exhibit oscillations due to the structural response to the forces being exerted.

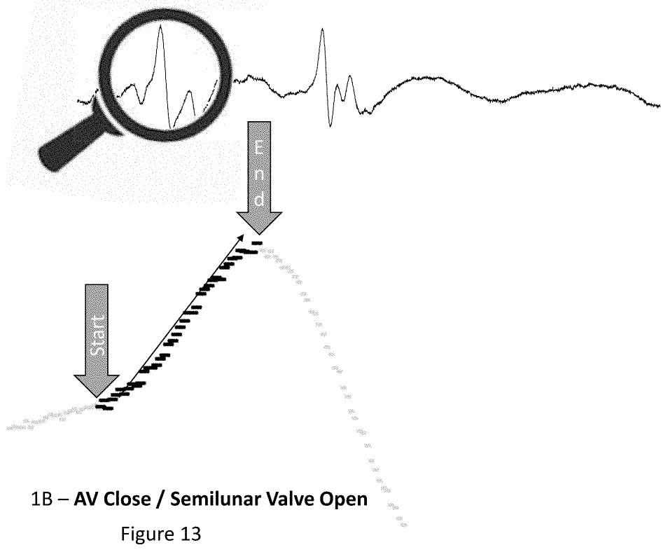

1B—AV Close/Semilunar Valve Open (FIG. 13)—This interval begins with the extreme positive going acceleration created by the surge of blood from the ventricles following the closure of the Arterial valves and the opening of the Semilunar valves. The rise is sharp with the highest acceleration rate, starting slightly negative in the quiescent range. The interval terminates at the highest peak of positive acceleration, and is typically the highest acceleration rate noted.

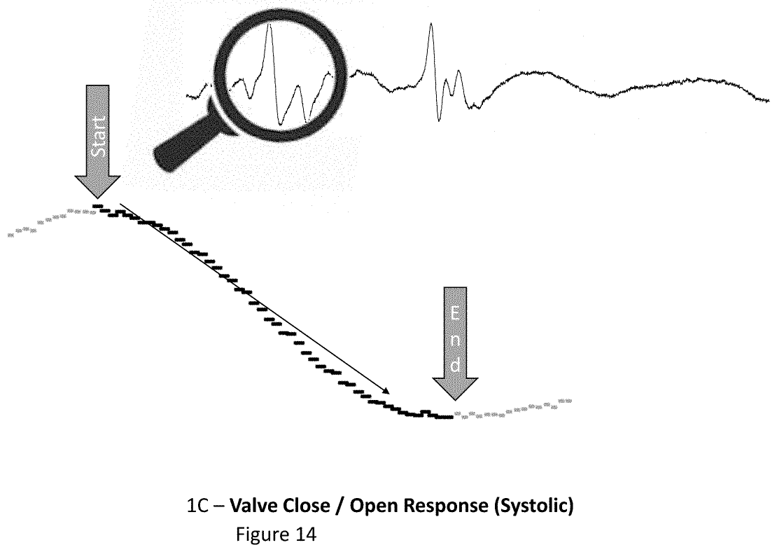

1C—Valve Close/Open Response (Systolic) (FIG. 14)—This interval starts at the first indication of a reversal of positive acceleration; a period of negative going acceleration “recovery” from the rapid positive acceleration. This interval ends when the negative going acceleration has fully countered the rapid and sharp acceleration of the previous interval. It is highly likely that this interval's actual termination point will be masked by oscillations that are separate and distinct from that of heart function, and will be closely related to the overall structural resonance. It is best to err on the side of “impact termination” which is the most negative point encountered from the previous interval's large positive going extreme.

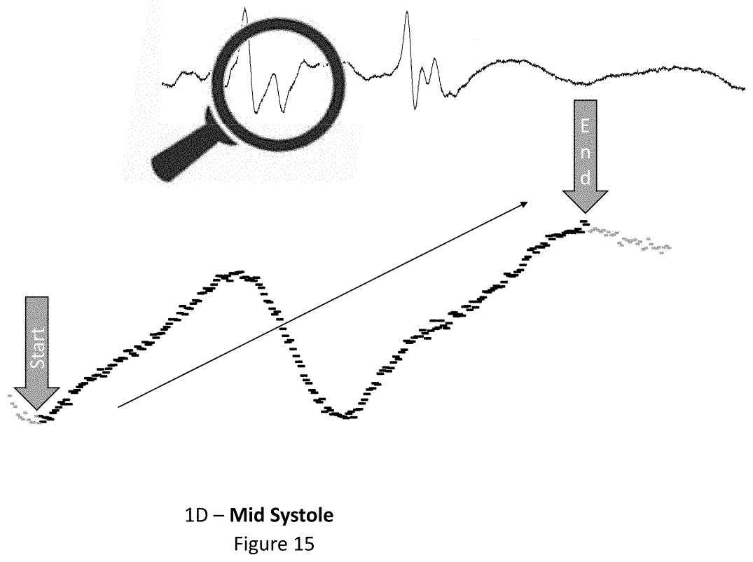

1D—Mid Systole (FIG. 15)—This interval is most often an interval of structural resonance oscillation of the overall structure. However, it is also a period of slow but moderately positive going recovery from the large negative going counter to the large positive extreme. Again, when the slope of the slow moderate positive going recovery changes to that of a much slower rise over time, that is the termination point of this interval.

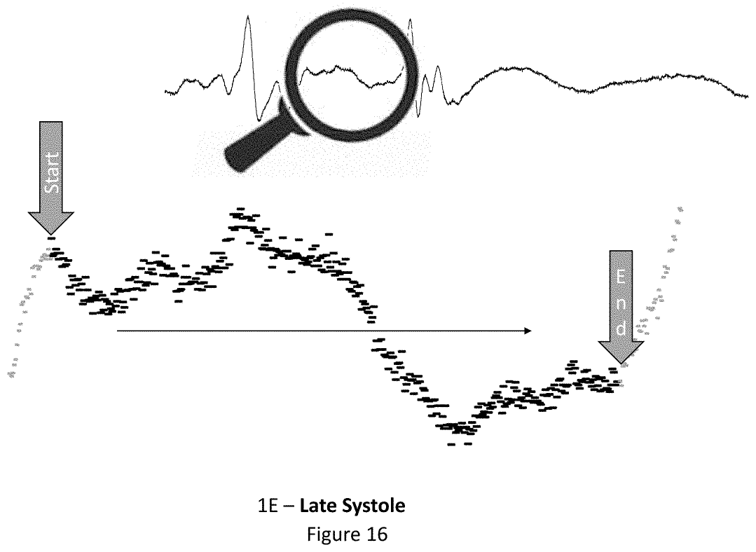

1E—Late Systole (FIG. 16)—The beginning of this interval is as stated above a change of the positive going slope of recovery. Because of the near zero acceleration rate it is highly susceptible to acceleration forces of blood pressure and respiration. It is best to select a heart-cycle for this interval that is at a respiration null and a period of stable blood pressure. Additionally, if the measurement was made during high heart rates, the task of this interval's identification is even more difficult. Fortunately, this interval's termination point is easily identified with the second high-positive going acceleration rate of the next interval.

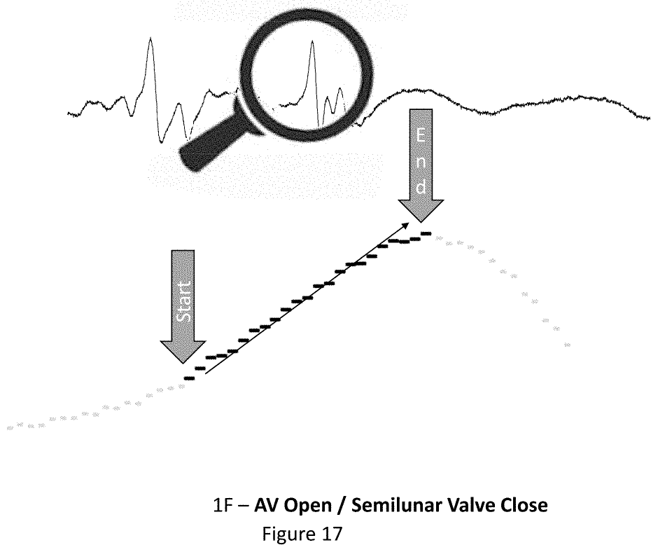

1F—AV Open/Semilunar Valve Close (FIG. 17)—This interval marks the beginning of the Diastolic period. The rapid positive acceleration force is typically the second highest positive peak in the acceleration waveform of the heart-cycle, when not influenced by respiration. This interval exhibits the highest positive-going rise only when the subject's physical activity is on the wane or intake respiration, as the static pressures of the body contributes to the apparent positive going force. Again, this interval (1F) like the first extreme positive going rise of acceleration rate (Interval 1C) begins at the first point of the sharp rise and terminates at the peak.

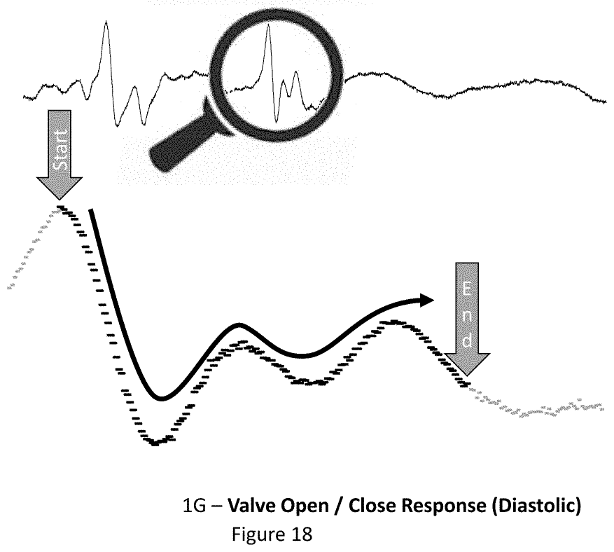

1G—Valve Open/Close Response (Diastolic) (FIG. 18)—Similar to the Systolic Valve Open/Close Response, this interval exhibits a large negative going acceleration rate starting at the typically second highest peak. However, it does not terminate at the most negative point, because the rapid negative going slope does not terminate at a point, it changes to that of an oscillation of the heart structure itself. The algorithm for this interval detection is dependent upon the oscillation's smooth transition from the negative slope to that of the related oscillation to differentiate it from the Systolic Valve Open/Close Response interval (Interval 1C). The termination of this interval is determined to be the point where the rate of oscillation acceleration differs in slope.

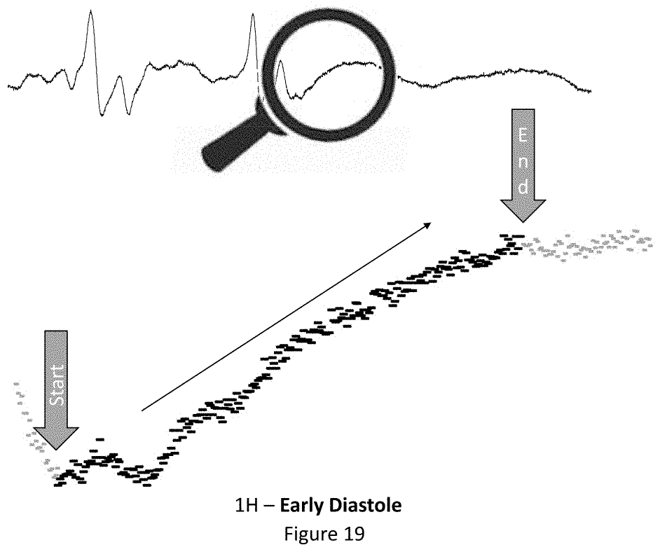

1H—Early Diastole (FIG. 19)—This interval as stated in interval 1G begins at the point where the slow positive acceleration rate changes. This will typically require an averaging of the cyclic oscillations of both the preceding interval and this interval. The interval terminates once more when the positive going acceleration rate changes again to the slope identified with the following interval.

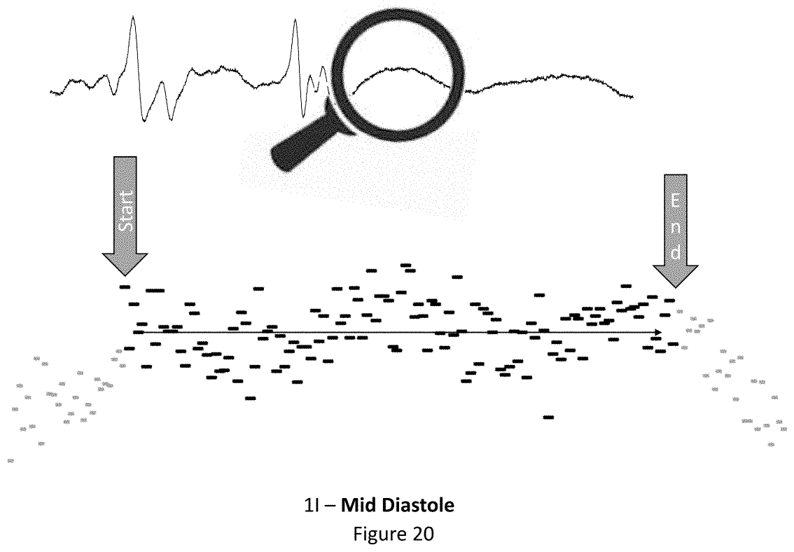

1I—Mid Diastole (FIG. 20)—This interval begins when acceleration very nearly does not exist. When the acceleration rate nears zero and flattens at that level, the beginning and end of that near zero acceleration mark the extents of this interval. The beginning is the point where it is no longer a positive acceleration from a negative region. The end is the point where the near zero acceleration starts to go negative. This interval is very dependent upon the subject being at rest, with a very steady normal heart-rate. If the subject has been active at all (high heart-rate) the Mid Diastole interval may not exist.

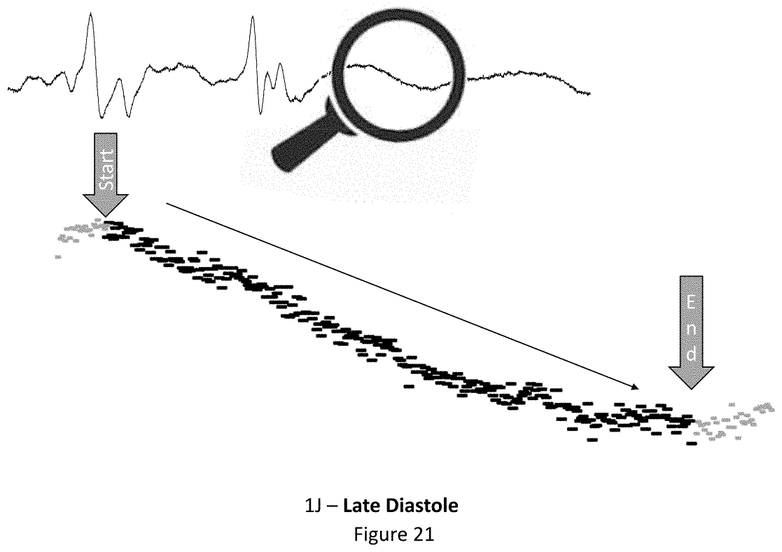

1J—Late Diastole (FIG. 21)—Again if the subject has been active; the beginning of this interval might show as a rapid change of acceleration rate from positive going to negative going. If this is true then the negative going slope will be sharper and lengthier, before it terminates with the changing of the heart function from a mode of muscular relaxation to that of expansion.

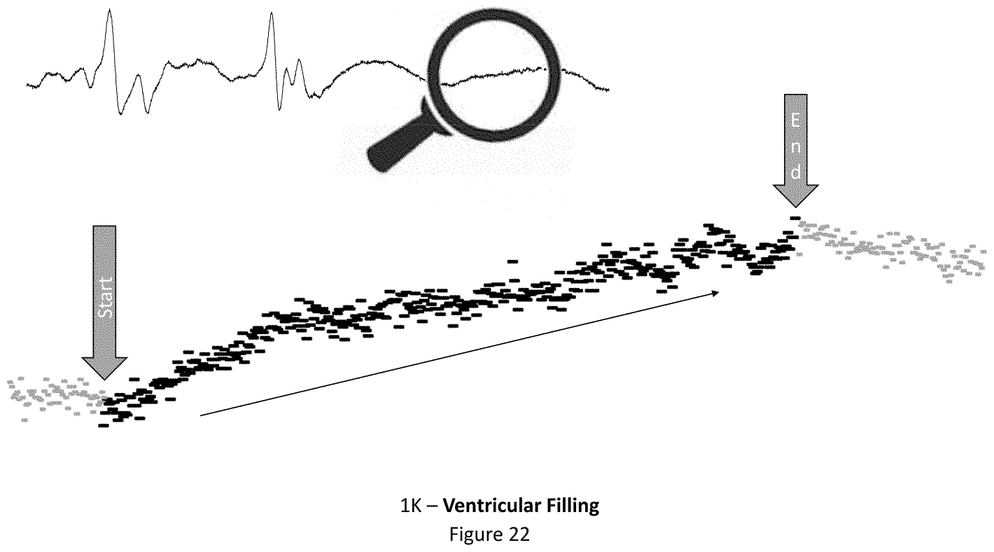

1K—Ventricular Filling (FIG. 22)—This interval shows a positive going transition through the quiescent acceleration rate region between extreme positive and extreme negative, as the heart is slowly expanding. It may show a slight curvature depending on the heart-rate. The termination of this interval is at the point where the slow gradual positive acceleration shows a change of “rate”. The heart will be expanding to a maximum and taking on a structural “stiffness” due to the blood's contribution to mass.

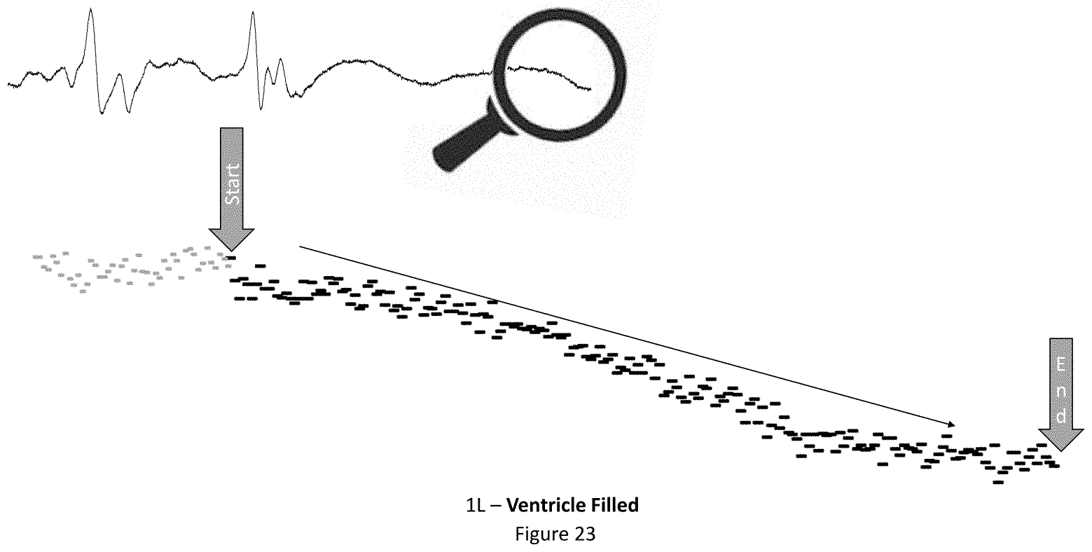

1L—Ventricle Filled (FIG. 23)—The beginning of this interval is the point where the “change of rate” reverses. The heart has expanded to its near maximum and the incoming blood has achieved its near maximum volume and influx pressure. The detected acceleration rate will show a negative trend and terminate when the Atrial Muscle begins to stiffen and the entire process repeats.

### Certain Examples of Use

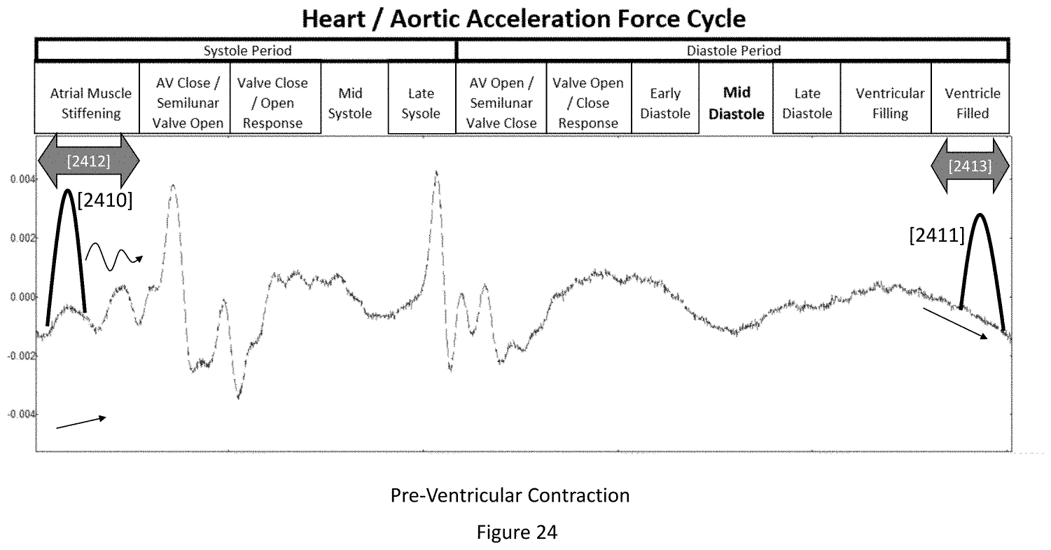

As an example of utilization, a non-critical heart function deviation is shown. The data of a normal heart-cycle with its markers and intervals is shown in FIG. 24 with two dark lines superimposed on the data trace [2410] and [2411. These two artificial deviations illustrate Pre-Ventricular Contractions (PVC). One feature of the system documented to date is that of the realization that PVC events with the sometimes-associated compensatory pause only take place when the PVC event takes place in the Atrial Muscle Stiffening interval [2412]. If the PVC event takes place during the Ventricle Filled interval [2413] there is very seldom an associated compensatory pause. It was not until this method of heart forcing function measurement was made possible that this critical timing issue was realized, as existing measurement systems could not accurately provide timing markers in that region.

In a second example of utilization, if this method is used in conjunction with pre-existing systems it is highly likely that Diastolic Dysfunction measurements of merit are possible, as well as other heart structure and capability characterizations can be made.

The method for non-invasive detection and classification of human cardiac forcing function markers and intervals, is a new method for the determination of actual heart functions in a subject measured and analyzed by the system described. These functions are characteristic of heart structure and muscle movement along with the flow of blood, where the flow of blood is a direct result of heart function. Each of the intervals and their markers are unique in terms of identification by algorithm or observation.

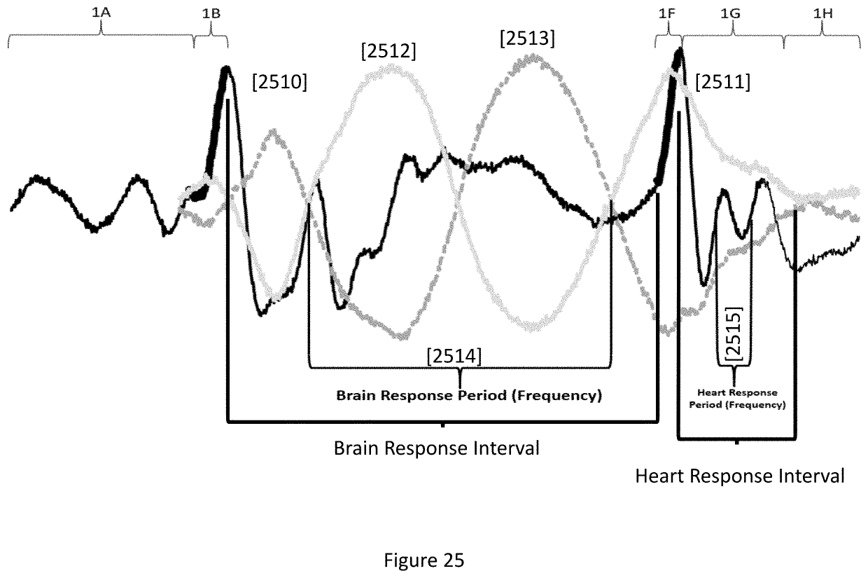

Systems capable of detecting response cranial acceleration signals may be enhanced with the heart function force additional information and can correct deficiencies in timing. The methods described can provide much more refined information with the division and precision of the heart force markers and intervals, as shown in FIG. 25. The association of force to the existing systems concentration on response can provide additional information such as localization of response functions regionally within the brain mass. Further to this would be surge of blood flow characteristics from the heart (arterial [2510]) and to the heart (venous [2511]).

Additionally, with the understanding of the forcing functions identified with this method, the basis of structural dynamics can be realized. Structures will respond to force input in predictable manners as factors of the Transfer function. Transfer function is simply defined as being equal to the output response divided by the input force, with specific attention paid to the characteristic's units of measurement, in this case a conversion of G's to Lbs. Force. Classic examples of impact forces are seen in this method's intervals of 1B and 1F. The following intervals 1C and 1G are often obfuscated by response functions, especially 1G; where the heart's own response is a part of the interval identification and separation from that of 1C. Never-the-less it is highly possible that transfer function descriptions of Dynamic Mass, Stiffness, Mobility and Compliance can be made possible with the proper attention to timing and signal phase relationships. With existing device enhancement refined with this method's concentration on force, it is highly likely that a heart's Diastolic Dysfunction could become a diagnostic measurement outcome of intervals 1F and 1G; as well as other heart function capabilities.

FIG. 25 illustrates the intervals of the Heart-Force described in intervals 1A through 1H, encompassing the Systole and Diastole high positive acceleration rates (1B and 1F) in the black data trace. The two light gray data traces show the Left [2512] and Right [2513] Temporal sensor's data over that same time frame as recorded by a system described in the Prior Art. A slight pre-amble and post cycle section of the two gray data traces show the lack of response before 1B and after 1F. Those two data traces illustrate the response function accelerations of the brain from the perspective of either side of the subject's head. Essentially the brain put in motion by the systolic force [2510]. It is important to note that this response oscillation of the brain as a direct function of the heart's systolic high positive acceleration force, and that response is then terminated with the high positive heart force associated with the high positive diastolic force [2511].

Take note of the period of the brain response [2514] to the systolic force, it is very different from that of interval 1G of the heart force [2515]. This difference in frequency is a function of the structural resonance. The much more distinct large oscillations are of a much lower frequency than those in interval 1G. This is additional evidence that the oscillation of interval 1G, which is a requirement of algorithm distinction to separate it from interval 1C, is that of heart structure resonance to the force of 1F.

Therefore, if the subject measured, is measured on a regular and repeating basis and that the data shows that the heart response period of interval 1G becomes higher in frequency over time, that subject is suffering from increasing Diastolic Dysfunction. Essentially the heart is becoming stiffer over time. With sufficient statistical data collected it may even be able to evaluate a subject's optimal heart structure with age and physical size. If that final conjecture is proven to be true, the method might be a predictor of heart structure issues as a function of the aging process, with potential drug and dietary recommendations for heart issue avoidance.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure. For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums).

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Java, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, optical line, or wireless communication network using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Conditional language, such as, among others, “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain embodiments require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general-purpose computer comprising one or more processors should not be interpreted as excluding other computer components, and may possibly include such components as memory, input/output devices, and/or network interfaces, among others.

While the above detailed description has shown, described, and pointed out novel features as applied to various embodiments, it may be understood that various omissions, substitutions, and changes in the form and details of the devices or processes illustrated may be made without departing from the spirit of the disclosure. As may be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A system for non-invasively determining information of a condition of the heart, comprising:
a first accelerometer adapted to be engaged externally at a position against the top of a patient's head to measure force from acceleration of the patient's skull caused by heartbeat-induced force through the sensitive pathway from heart to coronal peak of the skull, the first accelerometer configured to generate heartbeat force signal data responsive to said acceleration of the patient's upright skull caused by heartbeat-forcing function, the heartbeat force signal data corresponding to one or more heartbeat cycles;
a processing system coupled to the accelerometer, said processing system configured to
receive the heartbeat force signal data from the accelerometer,
determine, based at least in part from the heartbeat force signal data, a plurality of heartbeat cycle markers, heartbeat cycle intervals, and heartbeat force data of each interval, each interval representing a portion of a heartbeat cycle; and
determine information relating to a condition of the patient's heart by matching said one or more of the intervals of heartbeat cycle and the heartbeat force data with known signal data of abnormal heart conditions to thereby detect abnormal heart conditions of the patient non-invasively, and generate a display indicative of said abnormal heart conditions of the patient.

2. The system of claim 1, the position of the accelerometer against the top of a patient's head being at the peak and crest of the head of the patient when the head is held in an upright or vertical position.

3. The system of claim 1 or 2, wherein the position of the accelerometer is against the patient's head with sufficient static pressure to compress the patient's scalp skin and/or hair.

4. The system of any one of claims 1-3, wherein the position of the accelerometer is against the patient's head with sufficient pressure that the first accelerometer is considered in contact with the skull.

5. The system of any one of claims 1-4, wherein said accelerometer has a sensitivity to acceleration force of at least 500 mV/G

6. The system of claim 1, wherein the processing system is further configured to digitize the received heartbeat force data.

7. The system of claim 1, wherein determine information relating to a condition of the patient's heart includes generating and displaying one or more visualizations on a display that depict one or more of the heartbeat cycle intervals.

8. The system of claim 7, wherein the one for more visualizations include indications of abnormal conditions of the patient.

9. The system of claim 1, wherein the processing system determines the heartbeat cycle intervals based at least in part on determining markers indicting differing rates of change of acceleration trends of heartbeat force data within one heartbeat cycle

10. The system of claim 1, wherein determine information relating to a condition of the patient's heart includes comparing the determined heartbeat force data of at least one heartbeat cycle interval to previously collected heartbeat force data for a corresponding heartbeat cycle interval.

11. The system of claim 10, wherein the previously collected heartbeat force data is stored in a database.

12. The system of claim 10, wherein the processing system comprises a neural network system trained with previously collected heartbeat force data of heartbeat cycle intervals, and the processing system generates an input data set of the heartbeat force data related to the patient and compares the determined heartbeat force data to previously collected heartbeat force data using the neural network system.

13. The system of claim 1, wherein said processing system is further configured to analyze said signal data from three or more accelerometers, with one being located at the coronal peak by correlating to signal data from each of said plurality of accelerometers to determine a waveform signal common to the signal data from all of the three or more accelerometers.

14. The system of claim 1, wherein the interval of when the heart Atrial Muscle stiffening can be identified by a slow quiescent positive going slope, perhaps with some slight degree of oscillation.

15. The system of claim 1, wherein the interval where the heart's atrioventricular (AV) valves closing and Semilunar valve opening coupled with heart muscle contraction can be identified by detecting significant positive going forces which are exerted outwards from the heart to the lungs and body via the aortic arch, in the accelerometers sensitive pathway.

16. The system of claim 1, wherein the high positive acceleration of claim 5 changes to that of an extreme negative going recovery, marking the achievement of high blood pressure of the systolic period.

17. The system of claim 1, wherein the high negative acceleration presents an indication of response to the extreme preceding acceleration forces, and structural responses of the brain mass and surrounding matter can begin to oscillate.

18. The system of claim 1, wherein a slow gradual positive rise of acceleration rate can be a basis of continued response and recovery during an interval known as the Mid-Systole Interval.

19. The system of claim 1, wherein an interval of quiescent acceleration is exhibited, during which the heart has completed the surge of out-going blood, known as the Late Systole interval.

20. The system of claim 1, wherein the atrioventricular valves open and the semilunar valves close coupled with heart muscle relaxation can be identified by detecting an interval when significant positive going forces which are exerted upwards.

21. The system of claim 1, wherein the impact of the AV Valve opening and semilunar valve closing force has completed and a large negative going recovery interval begins, including the structural response of the heart mass not including the damping of contained blood.

22. The system of claim 1, wherein an interval of recovery from the large positive and negative acceleration rates known as the early diastole interval, where smaller more damped response oscillations of the heart muscle maybe present.

23. The system of claim 1, wherein an interval of quiescent slightly negative acceleration can be identified marking the interval called the mid-diastole.

24. The system of claim 1, wherein an interval of slightly negative going acceleration can be identified marking the interval called the late-diastole.

25. The system of claim 1, wherein a period of slowly positive going acceleration can be identified marking the interval of ventricular filling where blood is enlarging the physical size of the heart with an expanding force.

26. The system of claim 1, wherein a period of slowly negative going acceleration can be identified marking the interval where the ventricles have filled to their approximate maximum.

27. The system of claim 1, further comprising two or more other accelerometers adapted to be engaged externally at a position against the top of the patient's head to measure force from acceleration of the patient's skull caused by heartbeat-induced force through the sensitive pathway from heart to coronal peak of the skull.

28. A system for detecting heart conditions, comprising:
a processing system coupled to the accelerometer, said processing system configured to
receive heartbeat force signal data of a patient;
determine, based at least in part from the heartbeat force signal data, a plurality of heartbeat cycle markers, heartbeat cycle intervals, and heartbeat force data of each interval, each interval representing a portion of a heartbeat cycle; and
determine information relating to a condition of the patient's heart based at least in part on one or more of the intervals of heartbeat cycle and the heartbeat force data.

29. The system of claim 28, where the heartbeat force signal data is captured by a first accelerometer adapted to be engaged externally at a position against the top of a patient's head to measure force from acceleration of the patient's skull caused by heartbeat-induced force through the sensitive pathway from heart to coronal peak of the skull, the first accelerometer configured to generate heartbeat force signal data responsive to said acceleration of the patient's upright skull caused by heartbeat-forcing function, the heartbeat force signal data corresponding to one or more heartbeat cycles.

30. A method for non-invasively determining information of a condition of the heart based on heart force information, the method comprising:
collecting heartbeat force signal data using an accelerometer adapted to be engaged externally at a position against the top of a patient's head to measure force from acceleration of the patient's skull caused by heartbeat-induced force through the sensitive pathway from heart to coronal peak of the skull, accelerometer configured to generate heartbeat force signal data responsive to said acceleration of the patient's upright skull caused by heartbeat-forcing function, the heartbeat force signal data corresponding to one or more heartbeat cycles;

31. The system of claim 1, further comprising two or more other accelerometers adapted to be engaged externally at a position against the top of the patient's head to measure force from acceleration of the patient's skull caused by heartbeat-induced force through the sensitive pathway from heart to coronal peak of the skull.

32. A system for detecting heart conditions, comprising:
a processing system coupled to the accelerometer, said processing system configured to
receive heartbeat force signal data of a patient;
determine, based at least in part from the heartbeat force signal data, a plurality of heartbeat cycle markers, heartbeat cycle intervals, and heartbeat force data of each interval, each interval representing a portion of a heartbeat cycle; and
determine information relating to a condition of the patient's heart based at least in part on one or more of the intervals of heartbeat cycle and the heartbeat force data.

33. The system of claim 28, where the heartbeat force signal data is captured by a first accelerometer adapted to be engaged externally at a position against the top of a patient's head to measure force from acceleration of the patient's skull caused by heartbeat-induced force through the sensitive pathway from heart to coronal peak of the skull, the first accelerometer configured to generate heartbeat force signal data responsive to said acceleration of the patient's upright skull caused by heartbeat-forcing function, the heartbeat force signal data corresponding to one or more heartbeat cycles.

34. A method for non-invasively determining information of a condition of the heart based on heart force information, the method comprising:
collecting heartbeat force signal data using an accelerometer adapted to be engaged externally at a position against the top of a patient's head to measure force from acceleration of the patient's skull caused by heartbeat-induced force through the sensitive pathway from heart to coronal peak of the skull, accelerometer configured to generate heartbeat force signal data responsive to said acceleration of the patient's upright skull caused by heartbeat-forcing function, the heartbeat force signal data corresponding to one or more heartbeat cycles;
determining, based at least in part from the heartbeat force signal data, a plurality of heartbeat cycle markers, heartbeat cycle intervals, and heartbeat force data of each interval, each interval representing a portion of a heartbeat cycle; and
determining information relating to a condition of the patient's heart by matching said one or more of the intervals of heartbeat cycle and the heartbeat force data with known signal data of abnormal heart conditions to thereby detect abnormal heart conditions of the patient non-invasively, and generate a display indicative of said abnormal heart conditions of the patient.

35. A method for non-invasively determining information of a condition of the heart based on heart force information, the method comprising:
receiving heartbeat force signal data, the heartbeat force signal data collected using an accelerometer adapted to be engaged externally at a position against the top of a patient's head to measure force from acceleration of the patient's skull caused by heartbeat-induced force through the sensitive pathway from heart to coronal peak of the skull and to generate heartbeat force signal data responsive to said acceleration of the patient's upright skull caused by heartbeat-forcing function, the heartbeat force signal data corresponding to one or more heartbeat cycles;
determining, based at least in part from the heartbeat force signal data, a plurality of heartbeat cycle markers, heartbeat cycle intervals, and heartbeat force data of each interval, each interval representing a portion of a heartbeat cycle; and
determining information relating to a condition of the patient's heart based at least in part on one or more of the intervals of heartbeat cycle and the heartbeat force data.
